(19) United States
(12) Patent Application Publication
(10) Pub. No.: US 2026/0265666 A1
(43) Pub. Date: Sep. 10, 2026

(54) TEMPERATURE CONTROL SYSTEM FOR A LARGE-SCALE CELL CULTURE CHAMBER

(71) Applicant: CellFabs Inc., Tainan City (TW)

(72) Inventors: Yun-Shu Chiou, Tainan City (TW); Shang-Chih Lin, Tainan City (TW)

(73) Assignee: CellFabs Inc., Tainan City (TW)

(21) Appl. No.: 19/071,164

(22) Filed: Mar. 5, 2025

(51) Int. Cl. C12M 1/00; C12M 1/26; C12M 3/00; C12M 3/06

(52) U.S. Cl. CPC C12M 41/14; C12M 23/34; C12M 23/48; C12M 27/16; C12M 29/24; C12M 33/00

(57) ABSTRACT

A temperature control system for a large-scale cell culture chamber, comprising: A frame structure enclosed on the exterior by multiple isolation plates, with at least one culture rack installed inside the frame structure. The culture rack is sequentially provided with multiple bottom frame grids, multiple cell culture grids, and multiple top frame grids from the bottom to the top of the frame structure. Additionally, one side of the culture rack is adjacent to a thermal flow space. At least one heating module, each including multiple bottom-end fans, multiple top-end fans, and multiple heating elements. The bottom-end fans are fixed to the bottom frame grid, and the top-end fans are fixed to the top frame grid. The heating elements are distributed within the thermal flow space. Both the bottom-end fans and the top-end fans direct airflow toward the thermal flow space, ensuring that the hot air heated by the heating elements is evenly distributed across all cell culture grids, making the system suitable for large-scale cell culture chambers.

### BACKGROUND OF THE DISCLOSURE

### 1. Field of the Disclosure

The present invention relates to a large-scale cell culture chamber, and more particularly to a temperature control system for a large-scale cell culture chamber that achieves automated management through effective temperature control technology.

### 2. Description of the Prior Art

Conventional cell culture methods primarily utilize small-scale incubators to cultivate a single or a limited number of cell culture bottles. While this approach effectively controls environmental parameters in small-scale operations, it proves inefficient for large-scale cell production. When a large number of cells need to be cultured, additional incubators must be deployed, or multiple culture operations must be performed. This not only increases equipment costs but also adds operational complexity, making it difficult to meet the demands of modern biomedical applications and industrial-scale cell culture.

To overcome the limitations of small-scale incubators, the industry has proposed the use of large-scale cell culture chambers. While these chambers offer greater capacity and can accommodate multi-layered cell culture racks and a large number of cell culture bottles, they also introduce significant challenges. One major issue is the management of a vast number of cell culture bottles. Given the sheer quantity, efficient arrangement, retrieval, and monitoring require substantial manpower and time. This challenge is particularly evident during media exchange, inspection, or sample collection, where manual operations must be performed on each bottle individually. This not only increases labor intensity but also heightens the risk of errors and contamination. More critically, environmental control within a large-scale cell culture chamber, especially temperature uniformity, is difficult to ensure.

Cells are highly sensitive to temperature fluctuations, and even minor variations can significantly impact their growth and metabolism. In large-scale culture chambers, due to the vast space and complex structural design, heat distribution tends to be uneven. For instance, areas near the heating module may experience excessively high temperatures, whereas regions farther from the heat source may suffer from insufficient heating. Such temperature inconsistencies result in cell culture bottles being subjected to non-uniform environmental conditions, ultimately affecting overall culture efficiency and quality. Therefore, achieving automated management and precise temperature control in large-scale cell culture chambers is a key technical feature of the present invention.

In view of the foregoing, based on years of experience in the manufacturing, development, and design of related products, the inventor has conducted extensive research and meticulous evaluation to ultimately develop the present invention, which possesses significant practical utility.

### SUMMARY OF THE DISCLOSURE

The technical problem that the present invention aims to solve is to address the aforementioned shortcomings of existing technology by providing a temperature control system for a large-scale cell culture chamber.

A frame structure is enclosed on the exterior by multiple isolation plates. Inside the frame structure, at least one culture rack is installed. The culture rack is provided with multiple bottom frame grids at the lower end of the frame structure and multiple top frame grids at the upper end. Between the bottom frame grid and the top frame grid, multiple cell culture grids are arranged in a rectangular configuration. A thermal flow space is formed between the culture rack and the adjacent isolation plate. At least one heating module comprises multiple bottom-end fans, multiple top-end fans, and multiple heating elements. The bottom-end fans are fixed to the bottom frame grid, while the top-end fans are fixed to the top frame grid. The heating elements are distributed within the thermal flow space. Both the bottom-end fans and the top-end fans direct airflow toward the thermal flow space, ensuring that the hot air heated by the heating elements is evenly distributed to all cell culture grids.

A shaking device includes a shaking plate on which a cell culture bottle is placed. The bottom of the cell culture bottle is recessed with multiple positioning recesses, while the shaking plate is provided with multiple corresponding positioning projections. The engagement between the positioning recesses and the positioning projections prevents the cell culture bottle from detaching from the shaking plate.

The culture rack is equipped with multiple thermometers, and each cell culture bottle is equipped with a temperature sensor. The temperature sensor extends into the cell culture bottle to directly measure the temperature of the culture medium.

Two culture racks are arranged in parallel, forming an operation area between them. A transfer machine is installed in the operation area, and the transfer machine is provided with at least one clamping jaw, which grips the cell culture bottle. The frame structure includes a culture bottle window in the operation area, allowing the insertion and removal of cell culture bottles.

The transfer machine further comprises at least one transfer track, a steering shaft, and a translation shaft. The transfer track is fixed within the operation area, with one end positioned near the culture bottle window. The steering shaft is movably arranged on the transfer track, and the translation shaft is movably arranged on the steering shaft. The clamping jaw is movably mounted on the translation shaft.

Multiple convection fans and heating strips are fixed above the operation area. The convection fans face downward toward the operation area, directing the hot air generated by the heating strips toward the bottom-end fans.

At least one bottom-end heater is installed within the bottom frame grid, positioned between the thermal flow space and the bottom-end fan. At least one top-end heater is installed within the top frame grid, positioned between the thermal flow space and the top-end fan.

A guide plate is fixed at the interface between the bottom frame grid and the top frame grid within the thermal flow space. The guide plate is inclined to direct heated air into the thermal flow space. Additionally, a side plate is fixed to both sides of the culture rack to prevent heat from escaping.

The frame structure is equipped with multiple temperature-regulating fans within the thermal flow space. Each temperature-regulating fan is positioned directly opposite a corresponding cell culture grid, allowing for individual temperature regulation of each cell culture grid.

The primary objective of the present invention is to provide a frame structure with an internal culture rack. The culture rack includes a bottom frame grid at the lower end, a top frame grid at the upper end, and multiple cell culture grids arranged between them. One side of the culture rack is surrounded by a thermal flow space. The heating module includes bottom-end fans installed on the bottom frame grid and top-end fans installed on the top frame grid. The bottom-end fans and top-end fans are controlled to direct airflow into the thermal flow space. Together with the heating elements installed in the thermal flow space, the system ensures that the heated air is evenly distributed to each cell culture grid. This allows the cell culture bottles to be maintained within the optimal temperature range for cell culture, thereby achieving the temperature control requirements of large-scale cell culture.

Other objectives, advantages, and novel features of the present invention will become more apparent from the following detailed description and accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is a perspective view of the present invention;

FIG. 2 is a perspective view of the present invention with the isolation plate hidden;

FIG. 3 is a perspective cross-sectional view of the present invention in the horizontal direction;

FIG. 4 is a front schematic view of the present invention in the horizontal cross-section;

FIG. 5 is a perspective cross-sectional view of the present invention in the longitudinal direction;

FIG. 6 is a front schematic view of the present invention in the longitudinal cross-section;

FIG. 7 is an exploded perspective view of the shaking device and the cell culture bottle of the present invention;

FIG. 8 is a schematic diagram of hot air convection in the longitudinal cross-section of the present invention; and

FIG. 9 is a perspective schematic view of the transfer machine of the present invention.

### DETAILED DESCRIPTION

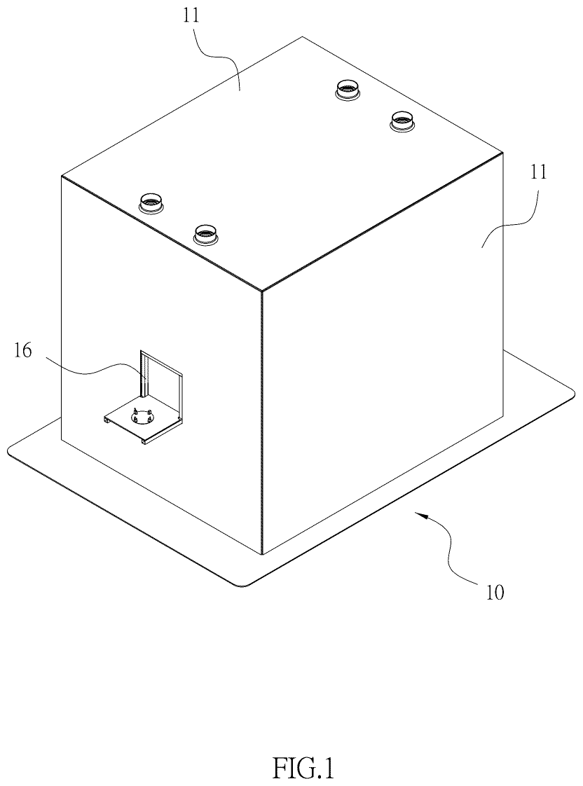

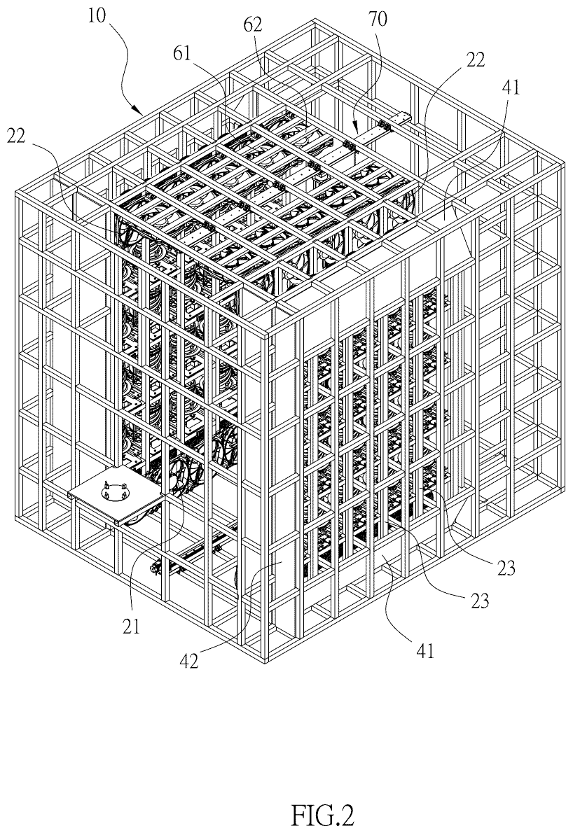

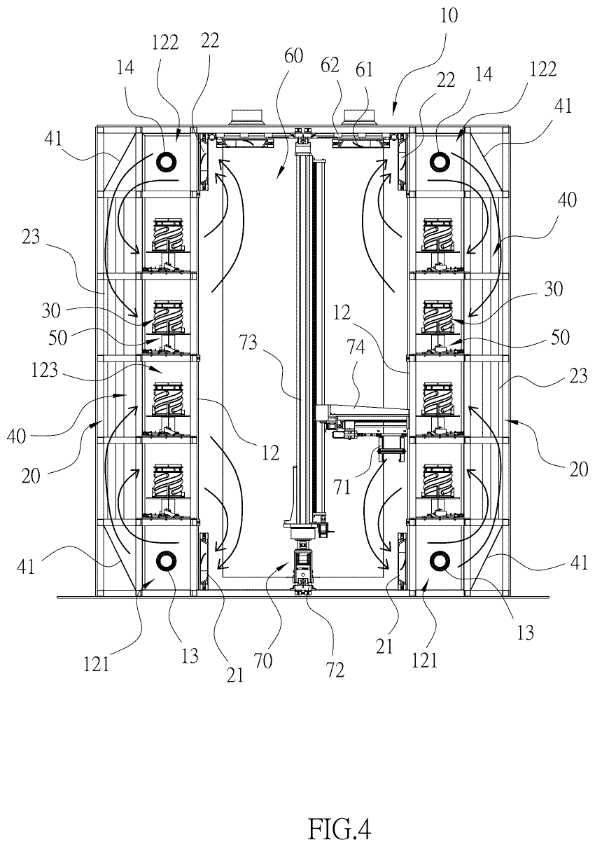

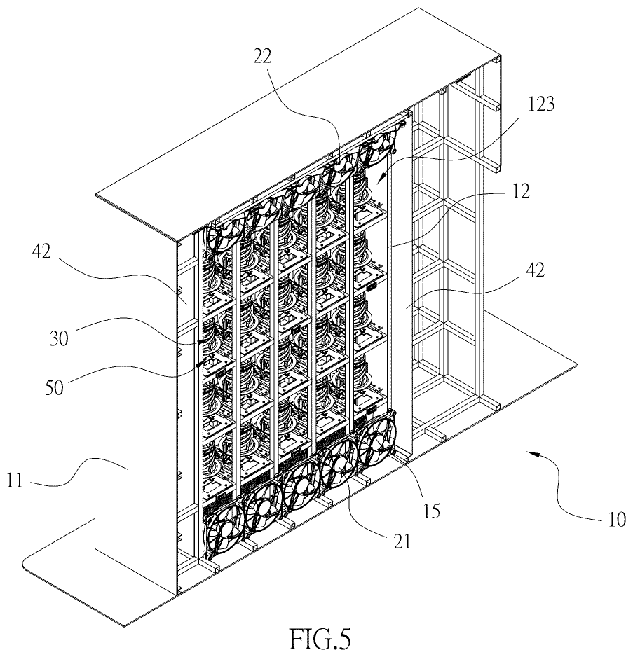

Referring to FIG. 1 through FIG. 6, a temperature control system for a large-scale cell culture chamber comprises a frame structure 10 and at least one heating module 20. The frame structure 10 is enclosed on the exterior by multiple isolation plates 11, which may be transparent thermal insulation glass. Inside the frame structure 10, at least one culture rack 12 is installed. The culture rack 12 is provided with multiple bottom frame grids 121 at the lower end of the frame structure 10 and multiple top frame grids 122 at the upper end. Between the bottom frame grid 121 and the top frame grid 122, multiple cell culture grids 123 are arranged in a rectangular configuration. The number of cell culture grids 123 in height is limited to four or six, while the width arrangement is unrestricted based on demand. The cell culture grids 123 are designed to hold a cell culture bottle 30. A thermal flow space 40 is formed between the culture rack 12 and the adjacent isolation plate 11, meaning that one side of the culture rack 12 allows thermal flow space 40 to communicate with the cell culture grids 123. The heating module 20 comprises multiple bottom-end fans 21, multiple top-end fans 22, and multiple heating elements 23. The bottom-end fans 21 are fixed to the bottom frame grid 121, while the top-end fans 22 are fixed to the top frame grid 122. The heating elements 23 are distributed within the thermal flow space 40. Both the bottom-end fans 21 and top-end fans 22 direct airflow into the thermal flow space 40, ensuring that the hot air generated by the heating elements 23 is evenly distributed to all cell culture grids 123. This configuration provides effective temperature control for cell culture, particularly in large-scale cell culture chambers, ensuring that each cell culture grid maintains an optimal temperature for cell growth.

Referring again to FIG. 1 through FIG. 6, the practical application of this invention is achieved by enclosing the frame structure 10 with isolation plates 11, forming a sealed space. The heating module 20 is installed inside the frame structure 10, enabling an internal circulation temperature control mechanism. When applied to cell culture, this design effectively reduces exposure to external contaminants. The large-scale cell culture chamber described in this invention accommodates more than twenty cell culture bottles 30 within the same space. Since these numerous cell culture bottles 30 are placed in different locations inside the chamber, a significant challenge arises in maintaining uniform temperatures across all cell culture bottles 30 or providing varied environmental temperatures as needed. To address this challenge, the frame structure 10 is equipped with the culture rack 12, which includes a bottom frame grid 121 at the lower end, a top frame grid 122 at the upper end, and multiple cell culture grids 123 positioned between them to hold the cell culture bottles 30. Furthermore, one side of the culture rack 12 is surrounded by the thermal flow space 40, which is connected to each bottom frame grid 121, top frame grid 122, and cell culture grid 123. The heating module 20 includes bottom-end fans 21 installed on the bottom frame grid 121 and top-end fans 22 installed on the top frame grid 122. Both the bottom-end fans 21 and top-end fans 22 are controlled to direct airflow into the thermal flow space 40. In combination with the heating elements 23 installed in the thermal flow space 40, the system ensures that the heated air is evenly distributed to each cell culture grid 123. This setup enables the cell culture bottles 30 to be maintained within an optimal temperature range for cell culture, thereby fulfilling the temperature control requirements of large-scale cell culture.

Referring again to FIG. 1 through FIG. 6, in the specific embodiment, the cell culture grids 123 of the culture rack 12 may be arranged in a matrix of four rows by five columns. The hot air blown by the bottom-end fan 21 tends to flow toward the lowest row, while the hot air blown by the top-end fan 22 tends to flow toward the highest row. Additionally, the hot air streams from the bottom-end fan 21 and the top-end fan 22 create an opposing airflow barrier in the middle of the thermal flow space 40, causing the redirected hot air to flow toward the second and third rows. This design ensures uniform temperature control for all cell culture bottles, achieving precise temperature regulation in a large-scale cell culture chamber. Furthermore, a guide plate 41 is fixed at the interface between the bottom frame grid 121 and the top frame grid 122 within the thermal flow space 40. The guide plate 41 is inclined to direct heated air into the thermal flow space 40. Additionally, both sides of the culture rack 12 are fixed with side plates 42 to prevent heat from escaping, thereby effectively controlling the direction of hot air circulation. Moreover, at least one bottom-end heater 13 may be installed within the bottom frame grid 121, positioned between the thermal flow space 40 and the bottom-end fan 21. Similarly, at least one top-end heater 14 may be installed within the top frame grid 122, positioned between the thermal flow space 40 and the top-end fan 22. When an increase in the internal temperature of the frame structure 10 is required, both the bottom-end heater 13 and the top-end heater 14 can be activated to rapidly raise the temperature. Alternatively, if the upper section of the frame structure 10 is warmer than the lower section, only the bottom-end heater 13 can be activated to balance the temperature difference within the frame structure 10. The heating element 23, bottom-end heater 13, and top-end heater 14 all utilize resistive heating for temperature control.

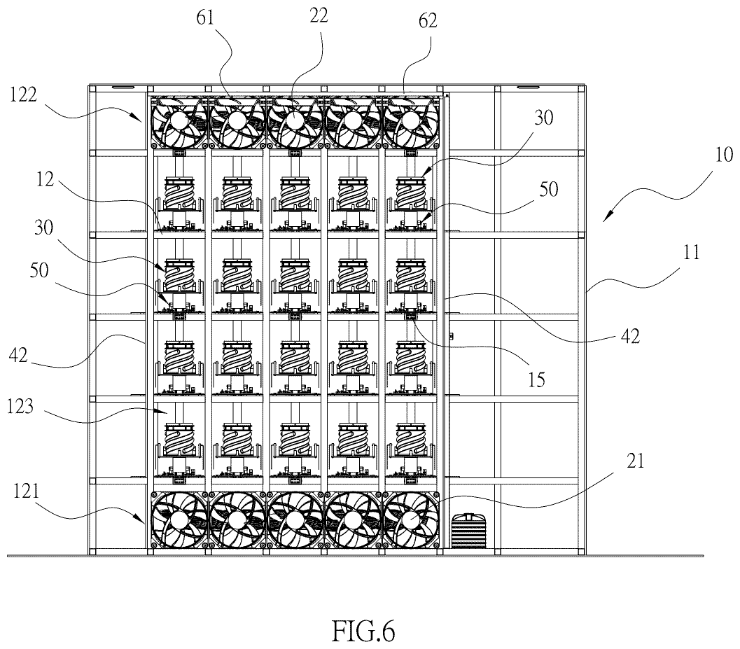

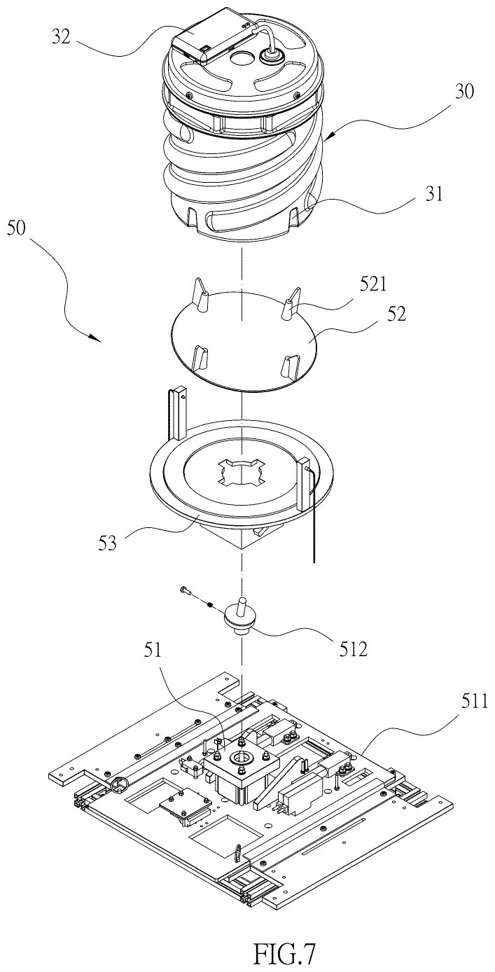

Referring to FIG. 6 and FIG. 7, the culture rack 12 is equipped with multiple thermometers 15, which measure actual temperatures at different positions inside the frame structure 10. Each cell culture bottle 30 is equipped with a temperature sensor 32, which extends into the cell culture bottle 30 to directly measure the temperature of the culture medium. This setup enables the collection of internal temperature data from both the frame structure 10 and the cell culture bottle 30. Based on the measurements from the thermometers 15, the overall temperature of the frame structure 10 can be automatically adjusted. Additionally, the temperature sensor 32 allows for independent temperature regulation of each cell culture bottle 30, thereby achieving automated temperature control.

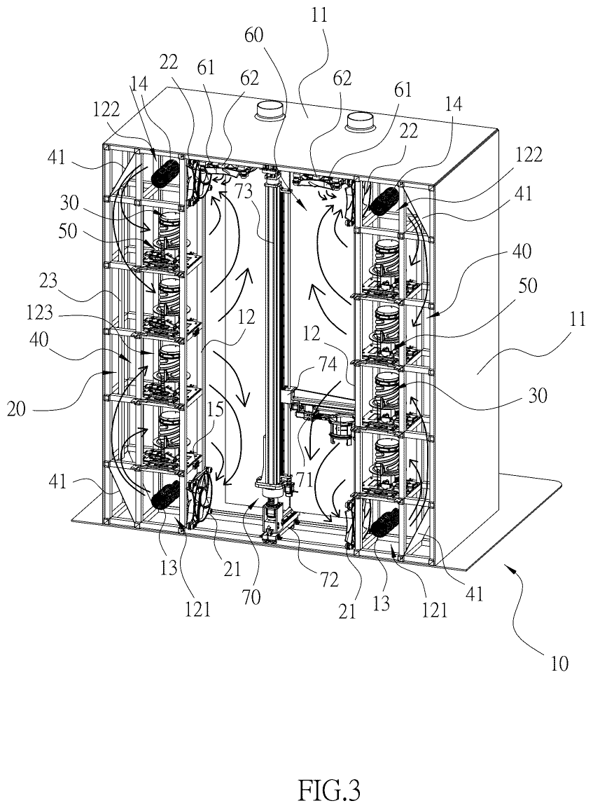

Referring to FIG. 3 and FIG. 7, each cell culture grid 123 is equipped with a shaking device 50. The shaking device 50 comprises an actuator 51, a shaking plate 52, and a limiting frame 53. The actuator 51 is fixed to a plate body 511 and is connected to an inclined seat 512. The bottom of the shaking plate 52 is positioned on the inclined seat 512, and the shaking plate 52 serves as a platform for holding the cell culture bottle 30. The limiting frame 53 is installed beneath the shaking plate 52. When the limiting frame 53 is lowered, the shaking plate 52 tilts, and the inclined seat 512 interacts with the shaking plate 52, causing the shaking plate 52 to rotate in an inclined circular motion. When the limiting frame 53 is raised, it supports the shaking plate 52 in a horizontal position. Additionally, when the limiting frame 53 is raised, the shaking plate 52 remains level, preventing the inclined seat 512 from driving the shaking plate 52. In this horizontal state, the cell culture bottle 30 can be easily retrieved. When the limiting frame 53 is lowered, it allows the inclined seat 512 to engage with the shaking plate 52. The actuator 51 then drives both the inclined seat 512 and the shaking plate 52, enabling the cell culture bottle 30 to perform a three-dimensional orbital shaking motion, thereby significantly enhancing the shaking effect. This technology incorporates the shaking device 50 within the cell culture chamber, continuously shaking the cell culture bottle 30 to prevent cell aggregation due to settling. Additionally, this movement increases the contact between cells and carbon dioxide, effectively enhancing cell activity. The shaking plate 52 of the shaking device 50 holds the cell culture bottle 30, which has multiple positioning recesses 31 recessed into its bottom surface. The shaking plate 52 is correspondingly designed with multiple positioning projections 521. The engagement between the positioning recesses 31 and the positioning projections 521 prevents the cell culture bottle 30 from detaching from the shaking plate 52, thereby improving the stability of the shaking device 50.

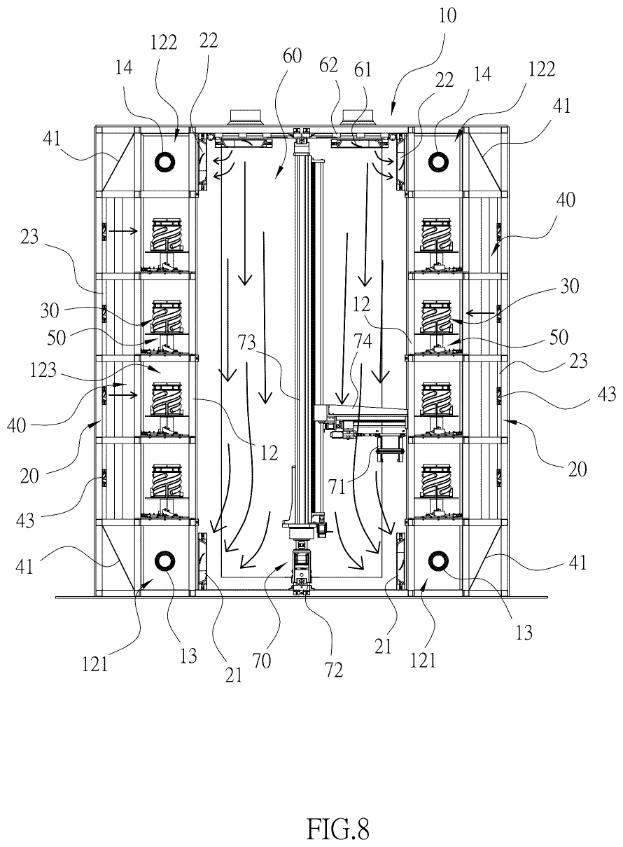

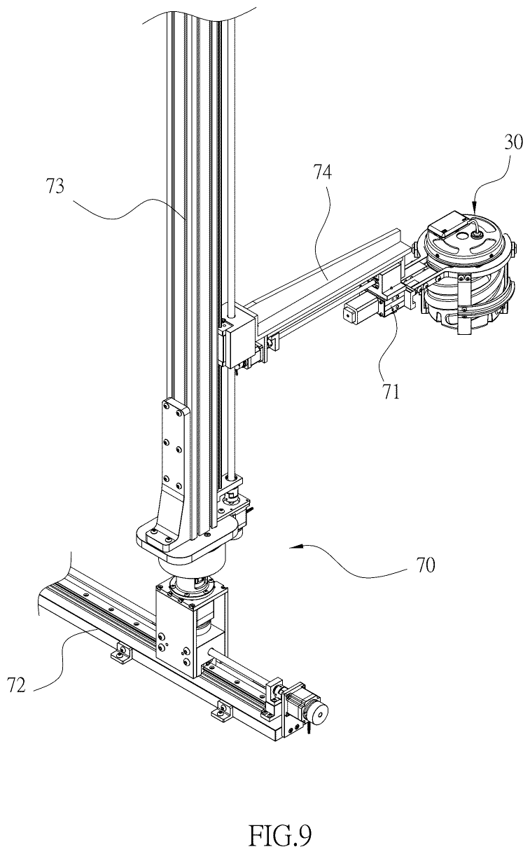

Referring to FIG. 8 and FIG. 9, in the specific embodiment, two culture racks 12 are arranged in parallel, forming an operation area 60 between them. A transfer machine 70 is installed within the operation area 60, and the transfer machine 70 extends at least one clamping jaw 71, which grips the cell culture bottle 30. Referring also to FIG. 1, the frame structure 10 is provided with a culture bottle window 16 in the operation area 60, allowing for the insertion and removal of the cell culture bottle 30 without requiring personnel to enter the frame structure 10. The transfer machine 70 further comprises at least one transfer track 72, a steering shaft 73, and a translation shaft 74. The transfer track 72 is fixed within the operation area 60, and preferably, two transfer tracks 72 are positioned at opposite locations on the upper and lower sections of the frame structure 10. One end of the transfer track 72 is positioned near the culture bottle window 16. The steering shaft 73 is movably arranged on the transfer track 72, allowing it to move back and forth along the transfer track 72, as well as perform axial rotation. The translation shaft 74 is movably arranged on the steering shaft 73, enabling it to reciprocate along the steering shaft 73. Additionally, the clamping jaw 71 is movably installed on the translation shaft 74, and the gripper 134 can move forward and backward along the translation shaft 133. This configuration allows the clamping jaw 71 to reach each cell culture grid 123, facilitating the gripping or placement of the cell culture bottle 30. The transfer machine 70 thereby supports automated operations such as media exchange, cell inspection, and cell harvesting, achieving automation management.

Referring to FIG. 8, multiple convection fans 61 and multiple heating strips 62 are fixed above the operation area 60. The convection fans 61 are directed downward toward the operation area 60, blowing the hot air generated by the heating strips 62 toward the bottom-end fan 21. When an increase in the internal temperature of the frame structure 10 is required, the convection fans 61 and heating strips 62 can be activated, providing additional heat sources. Conversely, when the upper section of the frame structure 10 is warmer than the lower section, the convection fans 61 can direct the accumulated hot air from the upper section to the lower section, effectively balancing the internal temperature of the frame structure 10. Furthermore, multiple temperature-regulating fans 43 are installed within the thermal flow space 40 of the frame structure 10. Each temperature-regulating fan 43 is positioned directly opposite a corresponding cell culture grid 123, allowing for individual temperature adjustments of each cell culture grid 123. If a specific cell culture bottle 30 is detected to be below the set temperature, the corresponding temperature-regulating fan 43 is activated to direct more hot air toward that particular cell culture bottle 30. This setup enables localized temperature modifications within the frame structure 10, accommodating cell culture bottles 30 with varying temperature requirements and achieving customized cell culture conditions.

The foregoing description is merely one preferred embodiment of the present invention and should not be construed as limiting its scope. Any modifications or variations made within the spirit and scope of the present invention, as defined in the appended claims, shall be encompassed within the patent coverage of the present invention.

What is claimed is:

1. A temperature control system for a large-scale cell culture chamber, comprising:
a frame structure, which is externally enclosed with multiple isolation plates, wherein at least one culture rack is erected inside the frame structure, the culture rack is provided with multiple bottom frame grids at the bottom end of the frame structure, the culture rack is provided with multiple top frame grids at the top end of the frame structure, multiple cell culture grids are arranged in a rectangular configuration between the bottom frame grid and the top frame grid, and a thermal flow space is formed between the culture rack and the adjacent isolation plate; and
at least one heating module, which includes multiple bottom-end fans, multiple top-end fans, and multiple heating elements, wherein the bottom-end fans are fixed to the bottom frame grid, the top-end fans are fixed to the top frame grid, the heating elements are distributed within the thermal flow space, and the bottom-end fans and top-end fans direct airflow into the thermal flow space, ensuring that the heated air from the heating elements flows evenly across all the cell culture grids.

2. The temperature control system for a large-scale cell culture chamber according to claim 1, wherein each cell culture grid is provided with a shaking device, the shaking device comprises an actuator, a shaking plate, and a limiting frame, the actuator is fixed to a plate body and connected to an inclined seat, the bottom of the shaking plate is positioned on the inclined seat, the limiting frame is disposed below the shaking plate, when the limiting frame moves downward, the shaking plate tilts, and the inclined seat interacts with the shaking plate to cause the shaking plate to rotate in an inclined orbital motion, and when the limiting frame moves upward, it supports the shaking plate in a horizontal position.

3. The temperature control system for a large-scale cell culture chamber according to claim 2, wherein a cell culture bottle is placed on the shaking plate of the shaking device, the bottom of the cell culture bottle is recessed with multiple positioning recesses, the shaking plate is correspondingly provided with multiple positioning projections, and the combination of the positioning recesses and the positioning projections prevents the cell culture bottle from detaching from the shaking plate.

4. The temperature control system for a large-scale cell culture chamber according to claim 3, wherein the culture rack is equipped with multiple thermometers, the cell culture bottle is equipped with a temperature sensor, and the temperature sensor penetrates into the cell culture bottle to directly measure the temperature of the culture medium.

5. The temperature control system for a large-scale cell culture chamber according to claim 3, wherein two culture racks are arranged in parallel, an operation area is formed between the two culture racks, a transfer machine is installed in the operation area, the transfer machine extends at least one clamping jaw to grip the cell culture bottle, the frame structure is provided with a culture bottle window at the operation area, and the frame structure allows the retrieval and placement of the cell culture bottle through the culture bottle window.

6. The temperature control system for a large-scale cell culture chamber according to claim 5, wherein the transfer machine further comprises at least one transfer track, a steering shaft, and a translation shaft, the transfer track is fixed within the operation area, one end of the transfer track is adjacent to the culture bottle window, the steering shaft is movably disposed on the transfer track, the translation shaft is movably disposed on the steering shaft, and the clamping jaw is movably disposed on the translation shaft.

7. The temperature control system for a large-scale cell culture chamber according to claim 5, wherein multiple convection fans and multiple heating strips are fixed above the operation area, the convection fans are oriented downward toward the operation area, and the convection fans direct the heated air generated by the heating strips toward the bottom-end fans.

8. The temperature control system for a large-scale cell culture chamber according to claim 1, wherein at least one bottom-end heater is installed within the bottom frame grid, the bottom-end heater is positioned between the thermal flow space and the bottom-end fan, at least one top-end heater is installed within the top frame grid, and the top-end heater is positioned between the thermal flow space and the top-end fan.

9. The temperature control system for a large-scale cell culture chamber according to claim 1, wherein a guide plate is fixed at the junctions of the bottom frame grid and the top frame grid within the thermal flow space, the guide plate is inclined to direct heated airflow into the thermal flow space, and side plates are fixed on both sides of the culture rack to prevent heated air from escaping from the sides of the culture rack.

10. The temperature control system for a large-scale cell culture chamber according to claim 1, wherein the frame structure is equipped with multiple temperature-regulating fans within the thermal flow space, each temperature-regulating fan is positioned directly in front of a cell culture grid, and the temperature-regulating fans regulate the temperature of individual cell culture grids.
